(19) United States
(12) Patent Application Publication
(10) Pub. No.: US 2017/0340409 A1
(43) Pub. Date: Nov. 30, 2017

(54) Apparatus and Method to Maintain Flow Through and Prevent Clogging of a Feeding Tube

(71) Applicant: Herb Lustberg, Kilingworth, CT (US)

(72) Inventor: Herb Lustberg, Kilingworth, CT (US)

(21) Appl. No.: 15/601,364

(22) Filed: May 22, 2017

Related U.S. Application Data
(60) Provisional application No. 62/340,597, filed on May 24, 2016.

(51) Int. Cl. A61B 90/70; B08B 9/032; A61J 15/00; B08B 9/043

(52) U.S. Cl. CPC A61B 90/70; B08B 9/0436; B08B 9/032; A61J 15/0069; A61B 2090/701

(57) ABSTRACT

A cleaning apparatus for an internal bore formed in a feeding tube has a shaft with a distal end and an opposing proximal end. The distal end terminates at a blunt tip and the proximal end terminates at a cap. The shaft has a length such that when inserted into the feeding tube, the cap aligns with a proximal end of the feeding tube and the blunt tip extends only partway out of a distal end of the feeding tube. At least one disc is mounted on the shaft adjacent the blunt tip. Each disc has a diameter equal to or greater than said first diameter of the internal bore so that tip portions of the disc deflect and travel along the internal bore of the feeding tube providing a squeegee-like cleaning action.

### CROSS-REFERENCE TO RELATED APPLICATIONS

This application claims the benefit under 35 U.S.C. §119(c) to U.S. Provisional Application No. 62/340,597, filed May 24, 2016, the entire contents of which are incorporated herein by reference in their entirety.

### BACKGROUND

When a person is unable to receive nutrients orally, a feeding tube may be employed. A distal end of the feeding tube may extend to the person's stomach, in which case it is referred to as a gastric tube. Alternatively, the distal end may extend to the person's small intestine, in which case it is referred to as a jejunal tube. An opposing proximal end of the feeding tube remains outside the person's body and accessible to medical personnel for introducing hydration, nutrients and medications, typically in the form of water-base viscous liquid suspension that many include crushed pills. If a residual portion of the viscous liquid suspension or crushed pills remains in the feeding tube, a clog may form, blocking the tube and preventing further nutrients from reaching the stomach or small intestine. Two conventional solutions to a clogged tube are: (1) clearing the clog and; (2) replacing the feeding tube.

Representative devices to clear a clog are disclosed in U.S. Pat. No. 6,725,492 to Moore et al.; United States Patent Application Publication 2009/0326513 A1 to Deutsch et al.; and United States Patent Application Publication 2011/0106019 A1 to Bagwell et al.

U.S. Pat. No. 6,725,492, entitled “Cleaning Brush for Medical Devices,” discloses a device having a distal tip formed from a metal or polymer. The distal tip leads a bristle brush through the feeding tube. The distal tip is disclosed as preventing the bristle portion from passing beyond the end of the feeding tube. US 2009/0326513 A1, entitled “Device for Placing an Occlusion Removing Structure in an Open-Ended Tubular Structure,” discloses a feeding tube de-clogger having a balloon mechanism to clear the end of the tube. US 2011/0106019 A1 titled, “Devices for Clearing Blockages in In-Situ Artificial Lumens,” discloses a clearing portion, such as a brush, driven by a repetitive motion portion, such as a motor. All three of U.S. Pat. No. 6,725,492; US 2009/0326513 A1 and US 2011/0106019 A1 are incorporated by reference herein in their entireties.

Alternatively, the clogged tube is removed and replaced with a new, unobstructed, feeding tube. United States Patent Application Publication 2014/0142552 A1 to Honig, entitled “Percutaneous Feeding Tube Including a Rescue Port,” discloses a feeding tube having an adjacent rescue port. When the feeding tube becomes clogged, a guide wire is inserted in the rescue port to retain the shape of the passage of the feeding tube. The clogged feeding tube is removed and a new feeding tube inserted utilizing the guide wire for alignment. US 2014/0142552 A1 is incorporated by reference herein in its entirety.

There remains a need for an apparatus and method that effectively prevents clogs from forming.

### BRIEF DESCRIPTION OF THE DRAWINGS

FIG. 1 is side perspective view of a feeding tube as known from the prior art.

FIG. 2 is a side perspective view of an apparatus for maintaining flow through a feeding tube as disclosed herein.

FIG. 3 is a cross-sectional view of a portion of the apparatus of FIG. 2 disposed within the feeding tube shown in FIG. 1.

FIG. 4 is a cross-sectional view of a portion of the apparatus of FIG. 2.

FIG. 5 is a cross-sectional view of a portion of the apparatus of FIG. 2 is accordance with a first embodiment disclosed herein.

FIG. 6 is a cross-sectional view of a portion of the apparatus of FIG. 2 is accordance with a second embodiment disclosed herein.

FIG. 7 is a side perspective view of an ENFit connection to be used in accordance with the invention.

FIG. 8 is a view of a feeding tube, in accordance with an embodiment disclosed herein.

### DETAILED DESCRIPTION

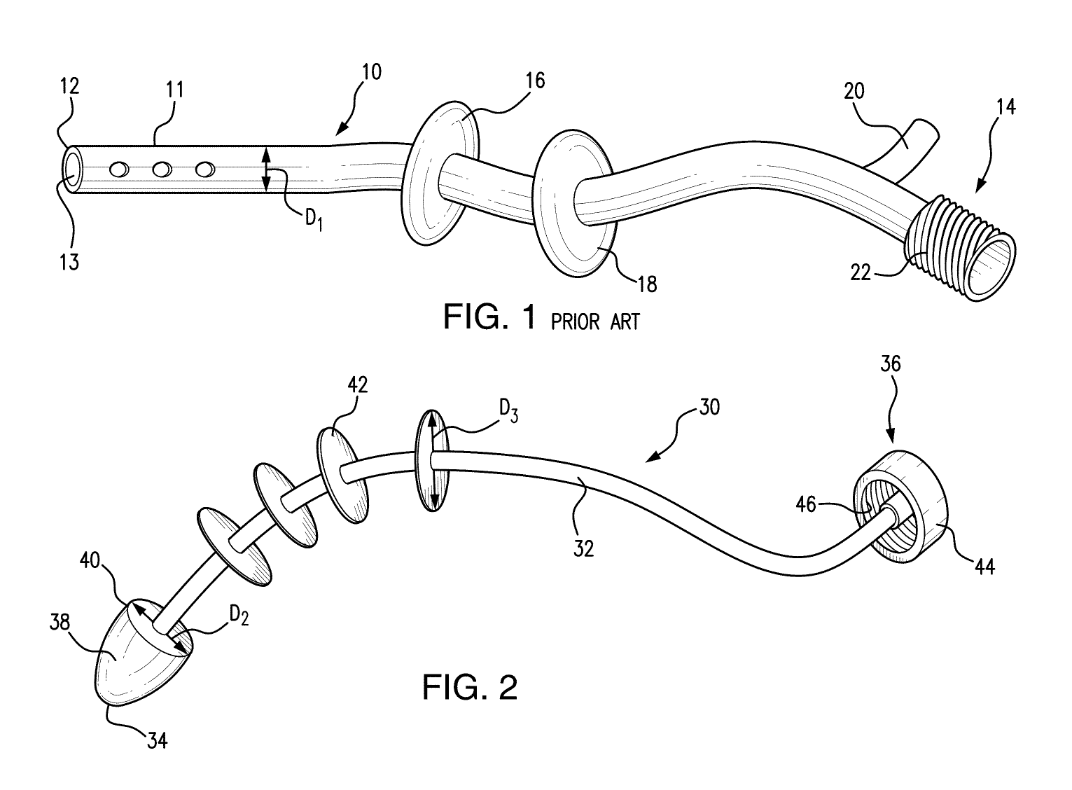

FIG. 1 is side perspective view of a feeding tube 10 as known from the prior art. The feeding tube 10 has a tube wall 11 with an outside diameter, D₁, and an internal bore 13. The feeding tube 10 has a distal end 12 and an opposing proximal end 14. At a point along the length of the feeding tube 10, an inflatable internal balloon 16 is located. An external bolster disc 18 is also located at some point along the length of the feeding tube 10.

When being installed into a patient, the inflatable internal balloon 16 is not inflated and its diameter is only slightly larger than the outside diameter of the feeding tube 10. An opening is formed through the patient's skin, typically in the area of the left upper quadrant of the abdomen. The distal end 12 is inserted into the patient up to a point dictated by the external bolster disc 18, so that the distal end is in the patient's stomach or small intestine. The inflatable balloon is then inflated via the balloon inflation port 20, so that the patient's skin and wall of the abdomen are between the inflatable internal balloon 16 and external bolster disc 18. As described below, the proximal end 14 preferably includes external threads 22.

Hydration, a suspension of nutrients and / or medicines in a liquid, or crushed pills may then be introduced into the patient by insertion into the feeding tube 10 at the proximal end 14. While the feeding tube 10 is typically flushed after each use, such as by water, clumps of solids frequently adhere to the tube wall 11 defining the inner bore 13. That is, clumps of solids frequently adhere to the inner wall of the feeding tube 10, ultimately causing clogs or disruptions.

As illustrated, in FIG. 2, in one embodiment of the invention, these clumps of solids are removed utilizing the inventive cleaning apparatus 30. The cleaning apparatus 30 includes a shaft 32 with a distal end 34 and an opposing proximal end 36. The distal end 34 terminates at a blunt tip 38.

It should be noted that blunt tip 38 is particularly formed such that, when the cleaning apparatus 30 passes through the feeding tube 10, the blunted tip 38 prevents the cleaning apparatus 30 from puncturing the wall of the feeding tube 10. Thus, the blunt tip 38 is specifically formed to remain in the lumen of the tube until it is entirely inserted into the tube. Additionally, blunt tip 38 is specifically formed to prevent any damage to the stomach or small intestine when completely inserted, such that, if the blunt tip 38 extends beyond the distal end of the feeding tube and into the stomach or small intestine, it will not puncture them.

Blunt tip 38 is generally conical in shape. In an embodiment, blunt tip 38 may increase gradually in diameter toward the proximal end 36. Thus, the end 40 of the blunt tip has a diameter, D₂, that is equal to, or about equal to, the diameter of the internal bore 13 of the feeding tube 10. In one embodiment, D₂ is minimally narrower when compared to the diameter of the internal bore 13. In another embodiment, D₂ is minimally wider when compared to the diameter of the internal bore 13. The blunt tip 38 is formed from any suitable material, such as plastic, that does not harm the tube, and that remains inert to the patient.

In one embodiment, disposed proximally to the end 40, as shown in FIG. 2, are one or more discs 42. Discs 42 are formed to remove adherent debris, tube feeds and crushed medication within the internal bore 13 of the feeding tube 10. Discs 42 wipe the inside of the tube, both upon entrance into the feeding tube and exit of the feeding tube. In an embodiment, the discs 42 are flush against the internal wall of the internal bore 13, thereby trapping debris.

In an embodiment, there are between two and six discs 42. In another embodiment, there may be four discs 42 located on shaft 32.

Discs 42 have a diameter, D₃, that may be greater than the diameter of the internal bore 13. In an embodiment, D₃ may be the same diameter as the internal bore 13. In another embodiment, D₃ may be smaller in diameter than internal bore 13.

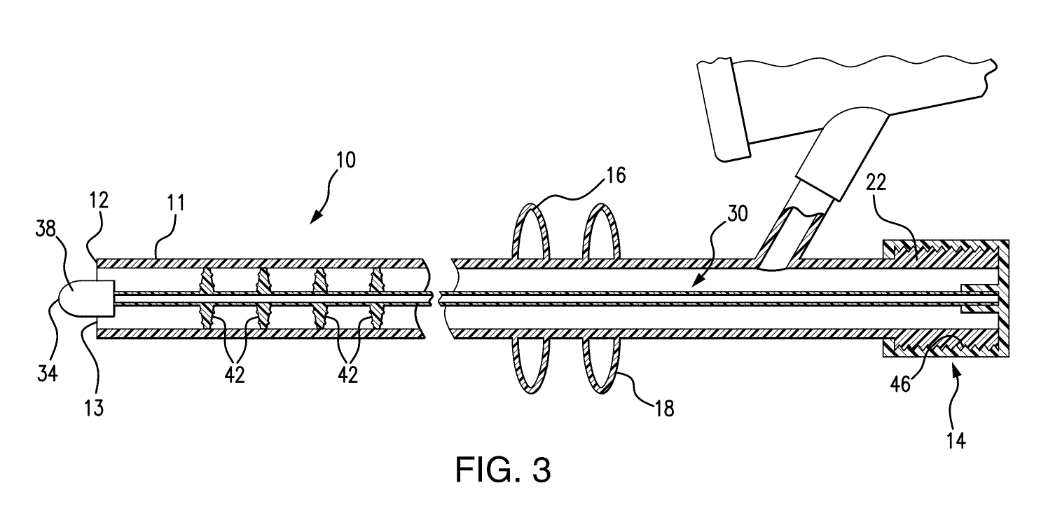

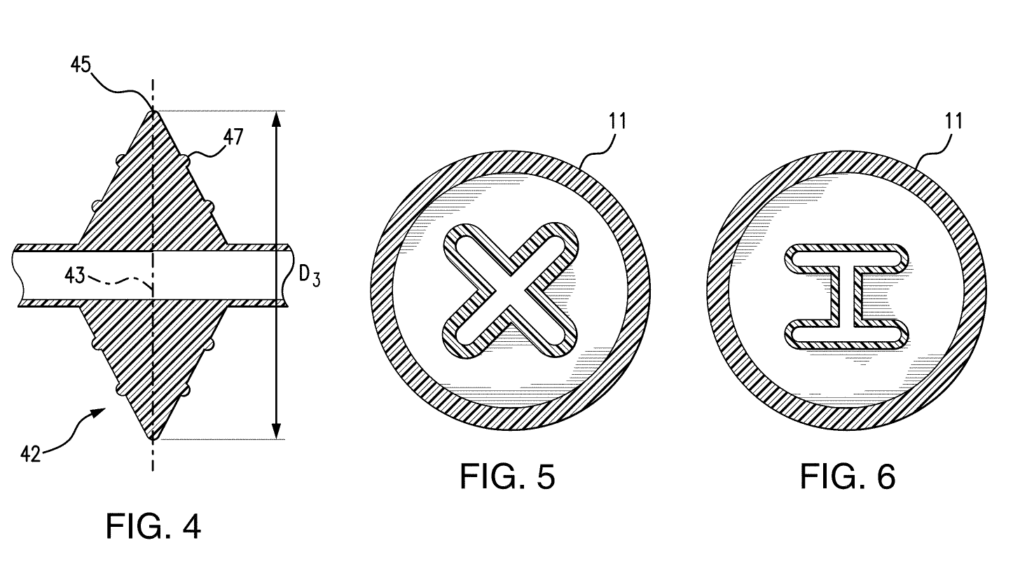

FIG. 3 illustrates the cleaning apparatus 30 disposed within the feeding tube 10. As shown, the discs 42 may contact the inner portion of tube wall 11, preventing debris from lingering in the tube, which would otherwise harden and the clog the feeding tube 10.

It should be noted that, in accordance with an embodiment, the cleaning apparatus 30 may be placed into the feeding tube 10 after each use, and may remain within the feeding tube 10 until the next use. This prevents stomach or intestinal contents from backing up into the feeding tube 10 between uses, thereby reducing clogging.

FIGS. 5-6 illustrate different embodiments of the discs 42. As illustrated, the discs 42 may be formed from any suitable material such as silicone, which provides the cleaning apparatus 30 with flexibility, while remaining strong and hypoallergenic.

In various embodiments, the diameter D₃ of the shaft 32 may be from very slightly less (nominally up to 0.005 inch less) to about 50%, by length, greater than the diameter of the internal bore 13, or from 20% to 30%, by length, greater than the diameter of the internal bore. It should be noted that, should the diameter be too large, it may make passage of a cleaning apparatus difficult, whereas, if the diameter is too small, it may result in incomplete cleaning or clearing of the feeding tube 10 lumen.

The discs 42 have a central portion 43 and tip portions 45. The tip portions taper to a point. This provides flexibility so that the tip portions deflect and travel along the wall of the internal bore, which provides a squeegee-like cleaning action. The discs 42 therefore provide a flexible mechanism for wiping away debris without damaging the feeding tube 10. Disc 42 includes projecting rings 47, which provide for strength. The strength may be provided similar to the strength of rolled sheet metal.

Referring back to FIG. 2, shaft 32 is formed from a suitable material, such as plastic, and is sufficiently rigid to drive the blunt tip 38 through the internal bore 13. Shaft 32 also remains sufficiently flexible such that it does not change the orientation of the feeding tube 10.

Referring now to FIGS. 5 and 6, shaft 32 is shown with an “X”-shape and an “H”-shape cross-section, respectively. These embodiments of the shaft provide longitudinal strength and retain flexibility to follow the curve of the tube. It should be noted, however, that shaft 32 may be formed in any cross-sectional shape.

Referring back to FIGS. 1 and 2, the length of the shaft 32 is sufficient such that when the proximal end 36 of the shaft is aligned with the proximal end 14 of the feeding tube 10, the end 40 of the blunt tip 38 is adjacent, but not beyond, the distal end 12 of the feeding tube.

The proximal end 36 of the cleaning apparatus terminates at a cap 44. Cap 44 includes internal threads 46 shaped to engage the external threads 22 of the feeding tube 10. In one embodiment, the feeding tube 10 and cleaning apparatus 30 are provided to the health care provider as an assembly, with the cleaning apparatus 30 inserted into the internal bore 13 and held in place by engagement of internal threads 46 and external threads 22.

In one embodiment, the cleaning apparatus 30 must be removed while the feeding tube is inserted into a patient, and it is replaced once the tube is in position. The inner cleaning apparatus 30 is removed for use of the feeding tube.

After use, preferably after each use, while any clumps remain moist and malleable, the cleaning apparatus is inserted into the internal bore 13 and pushed a sufficient distance so that the blunt tip 38 dislodges most clumps and pushes the material fainting the clumps into the patient. Any residual liquids or clumps are removed by the squeegee action of the discs 42. The cleaning apparatus 30 may then remain inserted inside the feeding tube 10 effectively sealing both ends of the feeding tube until it is time for the next use.

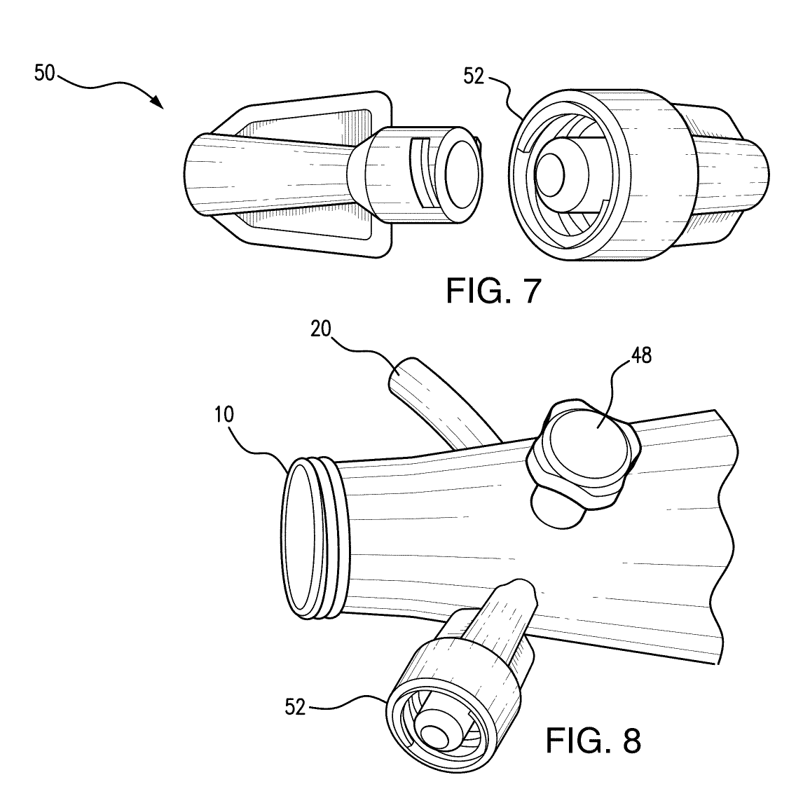

Referring now to FIG. 7, shown is pump set connector 50 and feeding port design that may be used in accordance with the invention. The pump set connector 50 is configured to be used with the ENFit connection system, thereby allowing the feeding tube to only connect to tube feeds. This prevents tube feeds from being delivered intravascularly.

Referring now to FIG. 8, illustrated is an embodiment of the invention with an external end of the feeding tube 10. The tube 10 allows the inner cleaning device 30 to pass through the interior bore of the tube 10, and securely screw into the external threads 22 via internal threads 46. The tube 10 may include a side port 20, for inflating the balloon.

The feeding tube 10 is ENFit compatible, and attaches the feeding tube 10 to a feeding formula container (not shown) via the EnFit connector 52, or any other suitable connector.

Mounted upon the feeding tube 10 is a stopcock 48. Stopcock 48 may be turned to not allow leakage. Alternatively, a cap (not shown) may be placed on the open end of the feeding tube 10 to prevent leakage.

In an embodiment, the stopcock 48 may be placed at the “Y” junction effectively direct flow into the patient when the ENFit connection is in use. Additionally, the stopcock 48 at the “Y” junction may prevent flow out of the external end of the tube 10. An ENFit connector is shown as connector 52.

When the cleaning apparatus 30 is desired to be used in the feeding tube 10, such as when not infusing feeds, the stopcock 48 allows the cleaning apparatus 30 to pass through the tube 10.

Although the disclosed subject matter has been described and illustrated with respect to embodiments thereof, it should be understood by those skilled in the art that features of the disclosed embodiments can be combined, rearranged, etc., to produce additional embodiments within the scope of the invention, and that various other changes, omissions, and additions may be made therein and thereto, without parting from the spirit and scope of the present invention.

What is claimed is:

1. A cleaning apparatus for an internal bore formed in a feeding tube, said internal bore having a first diameter, said cleaning apparatus comprising:
a shaft having a distal end and an opposing proximal end, the distal end terminating at a blunt tip and said proximal end terminating at a cap, said shaft having a length such that when inserted into said feeding tube, said cap aligns with a proximal end of said feeding tube and said blunt tip extends only partway out of a distal end of said feeding tube; and
at least one disc mounted on said shaft adjacent said blunt tip, each one of said discs having a diameter equal to or greater than said first diameter of the internal bore.

2. A cleaning apparatus for cleaning a feeding tube comprising:
a shaft including a distal end and a proximal end;
a plurality of discs mounted onto said shaft at the distal end;
wherein a diameter of the discs is greater than or equal to a diameter of an internal bore of the feeding tube.

3. The apparatus of claim 2, wherein the distal end of the shaft terminates at a blunt tip.

4. The apparatus of claim 3, wherein the blunt tip increases in diameter as it proceeds from the proximal end to the distal end.

5. The apparatus of claim 4, wherein the diameter of the blunt tip is narrower than the diameter of the internal bore of the feeding tube.

6. The apparatus of claim 4, wherein the diameter of the blunt tip is wider than the diameter of the internal bore of the feeding tube.

7. The apparatus of claim 2, wherein the proximal end terminates at a cap.

8. The apparatus of claim 7, wherein the cap includes internal threads shaped to engage external threads of the feeding tube.

9. The apparatus of claim 2, wherein the discs are formed flush against an internal wall of the internal bore.

10. The apparatus of claim 2, wherein the plurality of discs comprises four discs.

11. The apparatus of claim 2, wherein the shaft is formed from plastic.

12. The apparatus of claim 2, wherein the shaft is formed in a substantially X-shape.

13. The combination of a feeding tube and a cleaning apparatus for the feeding tube, comprising:
the feeding tube having a first length and an internal bore extending through the feeding tube the entire first length, said internal bore having a first diameter; and
the cleaning apparatus having:
a shaft with a distal end and an opposing proximal end, the distal end terminating at a blunt tip and said proximal end terminating at a cap, said shaft having a second length such that when inserted into said feeding tube, said cap aligns with a proximal end of said feeding tube and said blunt tip extends only partway out of a distal end of said feeding tube; and
at least one disc mounted on said shaft adjacent said blunt tip, each one of said discs having a diameter smaller than said first diameter of the internal bore.

14. The combination of claim 13 wherein a proximal end of said feeding tube has external threads that are engageable with internal threads formed on said cap.
